(19) United States
(12) Patent Application Publication
(10) Pub. No.: US 2021/0287773 A1
(43) Pub. Date: Sep. 16, 2021

(54) HYBRID COMPUTATIONAL SYSTEM OF CLASSICAL AND QUANTUM COMPUTING FOR DRUG DISCOVERY AND METHODS

(71) Applicant: HypaHub, Inc., Sunnyvale, CA (US)

(72) Inventors: Hugo Y. K. Lam, Sunnyvale, CA (US); Bayo Lau, San Jose, CA (US); Lijing Yao, Zhengzhou (CN)

(21) Appl. No.: 17/199,191

(22) Filed: Mar. 11, 2021

Related U.S. Application Data
(60) Provisional application No. 62/989,459, filed on Mar. 13, 2020.

(51) Int. Cl. G16H 20/10; G06F 9/48; G06F 9/38; G06N 10/00; G06F 9/30; G06N 20/00

(52) U.S. Cl. CPC G16H 20/10; G06F 9/4856; G06N 20/00; G06N 10/00; G06F 9/3005; G06F 9/3877

(57) ABSTRACT

A hybrid computational system of classical and quantum computing for drug discovery is provided for discovering drugs showing efficacy in affecting the behavior of a biological subject. The hybrid computational system of classical and quantum computing for drug discovery may include a computing environment, classical computing aspect, quantum computing aspect, compute workflow and machine learning operation. A method for discovering drugs showing efficacy in affecting the behavior of a biological subject using the hybrid computational system of classical and quantum computing for drug discovery is also provided.

### CROSS-REFERENCE TO RELATED APPLICATION

This application claims the priority from U.S. provisional patent application Ser. No. 62/989,459 filed Mar. 13, 2020. The foregoing application is incorporated in its entirety herein by reference.

### FIELD OF THE INVENTION

The present disclosure relates to a hybrid computational system of classical and quantum computing for drug discovery. More particularly, the disclosure relates to discovering drugs showing efficacy in affecting the behavior of a biological subject.

### BACKGROUND

Due to the rapid advancement of sequencing technologies, biomedical data are now being generated at an unprecedented rate. Today, sequencers shipped produce more than 100 petabytes of data per year, posing a significant challenge to computational biology. Using cloud computing in a distributed manner may help on horizontal scaling, but vertical scaling is still constrained by hardware factors and physical limits of Moore's Law, hampering the development of computationally intensive applications like Computer-Aided Drug Design (CADD).

Therefore, a need exists to solve the deficiencies present in the prior art. What is needed is a system and method for intelligently determining a computing platform for a workload between classical and quantum computing. What is needed is a hybrid system of classical and quantum computing to assist with drug discovery. What is needed is a hybrid system and method of classical and quantum computing to assist with determining efficacy of a drug for mutation in a biological subject. What is needed is a system and method to assist with advanced drug discovery via a network-connected computer device.

### SUMMARY

An aspect of the disclosure advantageously provides a system and method for intelligently determining a computing platform for a workload between classical and quantum computing. An aspect of the disclosure advantageously provides a hybrid system of classical and quantum computing to assist with drug discovery. An aspect of the disclosure advantageously provides a hybrid system and method of classical and quantum computing to assist with determining efficacy of a drug for mutation in a biological subject. An aspect of the disclosure advantageously provides a system and method to assist with advanced drug discovery via a network-connected computer device.

A system and method enabled by this disclosure may advantageously leverage quantum computers designed with quantum physics where it may not be constrained by Moore's Law to compliment the workflows previously performed via classical computing. Computing operations may be performed locally and/or remotely via a network, as cloud providers could offer access to quantum computing capacity. While quantum computing can be ideal for solving problems that are extremely hard for classical computing, such as simulating many-body dynamics as well as modeling the quantum states in a molecule with quantum-mechanical phenomena—superposition and entanglement, classical computing is still efficient and desirable for computations that are low in polynomial time. To solve these deficiencies between the capabilities of classical and quantum computers, especially in the field of drug discovery, the following disclosure describes a hybrid approach that combines both classical and quantum computing to achieve favorable results in biomedicine and other fields.

Accordingly, the disclosure may feature a hybrid computational system using classical computing and quantum computing for drug discovery to affect a behavior of a biological subject. The system may include a network over which data is communicated, a computing environment, and a memory. The computing environment may be accessible via the network. The computing environment may include a classical computing processor to perform the classical computing and a quantum computing processor to perform the quantum computing.

The memory may store machine-readable instructions to (a) receive parameters relating to the biological subject. The system may also (b) define a compute workflow to be performed by the computing environment by receiving a screening protocol relating to the biological subject. The compute workflow may include computing tasks. The system may also (c) connect to a repository via the network to retrieve data sets relating to the compute workflow. The system may also (d) selectively compare at least part of the computing tasks and at least part of the data sets for determining a likelihood of an advantage for the quantum computing compared to the classical computing for the computing tasks.

The system may (e) distribute a quantum computing task to be performed via the quantum computing if included by or recommended based on the likelihood of the advantage by the compute workflow. The system may also (f) distribute a classical computing task to be performed via the classical computing if included by or recommended based on the likelihood of the advantage by the compute workflow. The system may (g) perform the compute workflow via the computing environment to produce results. The system may also (h) organize the results returned by the computing environment to predict the drug discovery demonstrating a favorable efficacy to affect the behavior of the biological subject.

In another aspect, the compute workflow may include a machine learning operation to identify a drug having the favorable efficacy.

In another aspect, the machine learning operation may identify a probability of robustness in affecting the behavior of a mutation of the biological subject with approximately the favorable efficacy.

In another aspect, the machine learning operation may operate via ensemble machine learning including classical machine learning tasks performed via the classical computing and/or quantum machine learning tasks performed via the quantum machine learning.

In another aspect, the classical computing tasks may include molecular docking.

In another aspect, the classical computing tasks may include binding affinity prediction.

In another aspect, the classical computing tasks may include variant effect prediction.

In another aspect, the classical computing tasks may include lead search.

In another aspect, the quantum computing tasks may include structure analysis.

In another aspect, the quantum computing tasks may include molecular analysis.

In another aspect, an interface may be provided. The parameters may be received via the interface. At least some of the results may be presented via the interface.

In another embodiment, the disclosure may provide for a method for drug discovery to affect a behavior of a biological subject performed via a hybrid computational system using a computer environment to perform classical computing and quantum computing. The method may include (a) receiving parameters relating to the biological subject. The method may include (b) defining a compute workflow to be performed by the computing environment by receiving a screening protocol relating to the biological subject, the compute workflow comprising computing tasks. The method may include (c) retrieving data sets from a repository relating to the compute workflow.

The method may include (d) selectively comparing at least part of the computing tasks and at least part of the data sets for determining a likelihood of an advantage for the quantum computing compared to the classical computing for the computing tasks. The method may include (e) distributing a quantum computing task to be performed via the quantum computing if included by or recommended based on the likelihood of the advantage by the compute workflow. The method may include (f) distributing a classical computing task to be performed via the classical computing if included by or recommended based on the likelihood of the advantage by the compute workflow. The method may include (g) performing the compute workflow via the computing environment to produce results. The method may include (h) organizing the results returned by the computing environment to predict the drug discovery demonstrating a favorable efficacy to affect the behavior of the biological subject. The parameters may be received via an interface. At least some of the results may be presented via the interface.

In another aspect, the computing environment may be operable over a network. Data may be communicated via the network.

In another aspect, the compute workflow may include a machine learning operation. The method at step (g) may include (1) identifying a drug having the favorable efficacy via the machine learning operation.

In another aspect, step (g) may include (2) identifying a probability of robustness in affecting the behavior of a mutation of the biological subject with approximately the favorable efficacy.

In another aspect, step (g) may include (3) performing the machine learning operation via ensemble machine learning, wherein classical machine learning tasks are performed via the classical computing, and/or quantum machine learning tasks are performed via the quantum machine learning.

In another aspect, the classical computing tasks may include molecular docking, binding affinity prediction, variant effect prediction, and/or comprise lead search.

In another aspect, the quantum computing tasks may include structure analysis and/or molecular analysis.

In another embodiment, the disclosure may provide for a method for drug discovery to affect a behavior of a biological subject performed via a hybrid computational system using a computer environment to perform classical computing and quantum computing. The method may include (a) receiving parameters relating to the biological subject. The method may include (b) defining a compute workflow to be performed by the computing environment by receiving a screening protocol relating to the biological subject, the compute workflow comprising computing tasks. The method may include (c) retrieving data sets from a repository relating to the compute workflow.

The method may include (d) selectively comparing at least part of the computing tasks and at least part of the data sets for determining a likelihood of an advantage for the quantum computing compared to the classical computing for the computing tasks. The method may include (e) distributing a quantum computing task to be performed via the quantum computing if included by or recommended based on the likelihood of the advantage by the compute workflow. The quantum computing tasks may include structure analysis and/or molecular analysis. The method may include (f) distributing a classical computing task to be performed via the classical computing if included by or recommended based on the likelihood of the advantage by the compute workflow. The classical computing tasks may include molecular analysis, molecular docking, binding affinity prediction, variant effect prediction, and/or comprise lead search.

The method may include (g) performing the compute workflow via the computing environment to produce results, further comprising. The method may include (1) identifying a drug having a favorable efficacy in affecting the behavior of the biological subject via the machine learning operation. The method may include (2) identifying a probability of robustness in affecting the behavior of a mutation of the biological subject with the favorable efficacy. The method may include (h) organizing the results returned by the computing environment to predict the drug discovery demonstrating the favorable efficacy.

In another aspect, the parameters may be received via an interface. At least some of the results may be presented via the interface.

Terms and expressions used throughout this disclosure are to be interpreted broadly. Terms are intended to be understood respective to the definitions provided by this specification. Technical dictionaries and common meanings understood within the applicable art are intended to supplement these definitions. In instances where no suitable definition can be determined from the specification or technical dictionaries, such terms should be understood according to their plain and common meaning. However, any definitions provided by the specification will govern above all other sources.

Various objects, features, aspects, and advantages described by this disclosure will become more apparent from the following detailed description, along with the accompanying drawings in which like numerals represent like components.

### BRIEF DESCRIPTION OF THE DRAWINGS

FIG. 1 is a block diagram view of an illustrative computing platform, according to an embodiment of this disclosure.

FIG. 2 is a block diagram view of an illustrative compute workflow, according to an embodiment of this disclosure.

FIG. 3 is a block diagram view of an illustrative computerized device upon which classical computing operations may be performed, according to an embodiment of this disclosure.

FIG. 4 is a perspective rendered view of an illustrative crystal structure of a known virus inhibitor used during an experimental trial, according to an embodiment of this disclosure.

FIG. 5 is a chart view of an illustrative binding affinity of a screened virus used during an experimental trial, according to an embodiment of this disclosure.

FIG. 6 is a chart view of an illustrative Venn diagram showing results of a screened virus used during an experimental trial, according to an embodiment of this disclosure.

### DETAILED DESCRIPTION

The following disclosure is provided to describe various embodiments of a hybrid computational system of classical and quantum computing for drug discovery. Skilled artisans will appreciate additional embodiments and uses of the present invention that extend beyond the examples of this disclosure. Terms included by any claim are to be interpreted as defined within this disclosure. Singular forms should be read to contemplate and disclose plural alternatives. Similarly, plural forms should be read to contemplate and disclose singular alternatives. Conjunctions should be read as inclusive except where stated otherwise.

Expressions such as “at least one of A, B, and C” should be read to permit any of A, B, or C singularly or in combination with the remaining elements. Additionally, such groups may include multiple instances of one or more element in that group, which may be included with other elements of the group. All numbers, measurements, and values are given as approximations unless expressly stated otherwise.

For the purpose of clearly describing the components and features discussed throughout this disclosure, some frequently used terms will now be defined, without limitation. The term classical computing, as it is used throughout this disclosure, is defined as binary computing performed by manipulating bits between 0's and 1's. The term quantum computing, as it is used throughout this disclosure, is defined as computing that uses superposition, entanglement, tunneling, fluctuation, topology, and/or other quantum phenomena, which may benefit from models such as photonic quantum computing, nonlinear optical computing, Boson sampling, quantum logic gates, quantum Turing processes, adiabatic quantum computing, quantum annealing, ion traps, topological quantum computing, and/or other models and processes relating to manipulating and evaluating a state of quantum bits to solve computational problems. The term Moore's Law, as it is used throughout this disclosure, is defined as an observation that a number of transistors in an integrated circuit tends to double about every two years.

The term biomedical informatics, as it is used throughout this disclosure, is defined as a field of science and engineering that uses computational resources to improve provision of healthcare. The term drug discovery, as it is used throughout this disclosure, is defined as relating to discovery of new drugs and medicines, which may be assisted by computers. The term computer-aided drug design, as it is used throughout this disclosure, is defined as a process of finding new drugs based on knowledge of a biological subject, including various biological entities and pathogenic targets, which may be assisted by computers.

The term metadata, as it is used throughout this disclosure, is defined as data providing information relating to other data, which may be embedded in files or other data. The term repository, as it is used throughout this disclosure, is defined as a database of digital content or other data storage structure. The term database, as it is used throughout this disclosure, is defined as a collection of data organized for search and retrieval, which may include data storage structures such as a single database, network of databases, repository, file system, network file system, block storage, data storage managed by a database management system, and/or other data storage structures that would be apparent to those of skill in the art, without limitation. The term versioning, as it is used throughout this disclosure, is defined as management of files, computer programs, and other digital information with respect to changes.

The term molecular dynamics, as it is used throughout this disclosure, is defined as a computer simulation that can be used to analyze the physical movements of molecules and atoms. The term lead compounds, as it is used throughout this disclosure, is defined as a chemical compound possessing a pharmacological or biological effect, which may be therapeutically useful.

The term biological subject, as it is used throughout this disclosure, is defined as a target of a drug, which may affect the behavior of a molecule or entity associated with such target, for example including bacterium, virus, fungus, other microorganism, cells, subcellular molecules, organelles, proteins, receptors, enzymes, genes, DNA segments, RNA segments, and other subjects that can cause disease, cancer, cellular mutation, or other adverse biological function that would be appreciated by those of skill in the art. The term genomic information, as it is used throughout this disclosure, is defined as information focusing on structure, function, evolution, mapping, and editing of genomes. The term virus, as it is used throughout this disclosure, is defined as a biological subject that replicates in the cells of living organisms. The term mutation, as it is used throughout this disclosure, is defined as an alteration in the genome of a biological subject, such as a mutation to a genetic code. The term protein structure, as it is used throughout this disclosure, is defined as an arrangement of atoms in an amino-acid chain molecule, which may be at least partially folded to create a three-dimensional structure.

The term molecular docking, as it is used throughout this disclosure, is defined as binding of one molecule to another, which may be useful in structure-based drug design. The term binding affinity, as it is used throughout this disclosure, is defined as a likelihood that molecules will bind with a binding site, such as a receptor, which may be indicative of an attractive force. The term pharmacophore modeling, as it is used throughout this disclosure, is defined as modeling of an abstract description of a molecular feature to predict supramolecular interactions with a biological subject, such as a biological entity or pathogenic target, without limitation, and to affect a biological response. The term binding sites, as it is used throughout this disclosure, is defined as a region on a protein or other molecule that binds to another molecule, for example, with specificity.

The term machine learning, as it is used throughout this disclosure, is defined as operation of computer machine instructions to improve predictive capabilities over time and iteration. The term ensemble approach or ensemble method, as it is used throughout this disclosure, is defined as use of multiple machine learning approaches or methods to increase predictive performance. The term parallelized processing, as it is used throughout this disclosure, is defined as performing multiple computational operations substantially simultaneously, which may include a mix of classical and quantum computational operations. The term classifier, as it is used throughout this disclosure, is defined as a function that performs classification, such as by mapping input data into a category. The term robustness, as it is used throughout this disclosure, is defined as an ability to maintain an approximately similar efficacy when faced with changes in conditions, such as with a drug remaining effective despite a mutation in a target biological subject.

Various aspects of the present disclosure will now be described in detail, without limitation. In the following disclosure, a hybrid computational system of classical and quantum computing for drug discovery will be discussed. Those of skill in the art will appreciate alternative labeling of the hybrid computational system of classical and quantum computing for drug discovery as a hybrid system of classical and quantum computing for advanced biomedical information, hybrid computing system, efficient biomedical information computing platform, drug discovery system, mutation prediction platform, the invention, or other similar names. Similarly, those of skill in the art will appreciate alternative labeling of the hybrid computational system of classical and quantum computing for drug discovery as a hybrid computing organization method, drug discovery method, computing classification and drug discovery method, method, operation, the invention, or other similar names. Skilled readers should not view the inclusion of any alternative labels as limiting in any way.

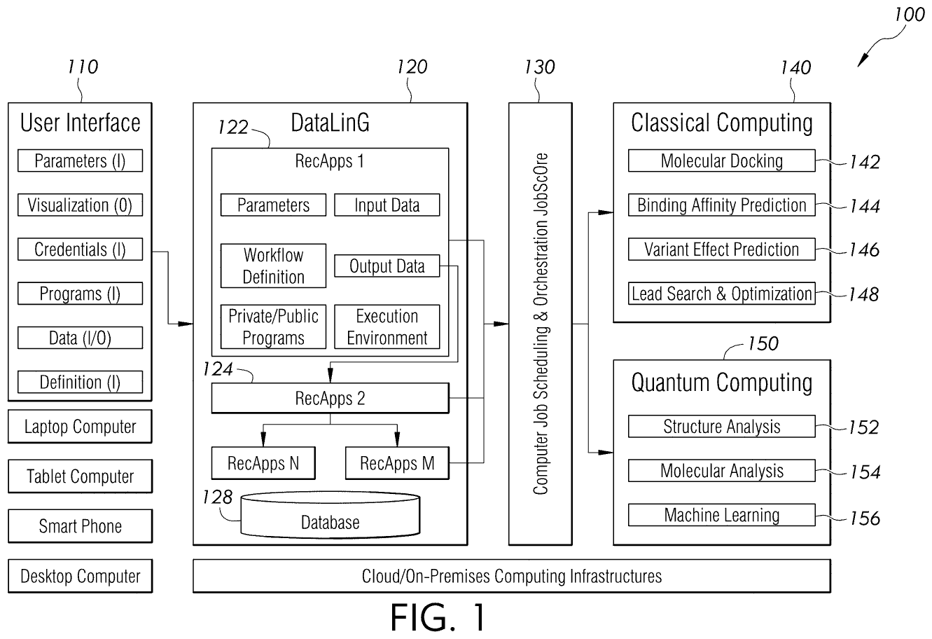

Referring now to FIGS. 1-6, an illustrative hybrid computational system of classical and quantum computing for drug discovery will now be discussed in more detail. The hybrid computational system 100 of classical and quantum computing for drug discovery may include a computing environment, classical computing aspect 140, quantum computing aspect 150, compute workflow, machine learning operations 149, 156, and additional components that will be discussed in greater detail below. The hybrid computational system of classical and quantum computing for drug discovery may operate one or more of these components interactively with other components for discovering drugs showing efficacy in affecting the behavior of a biological subject.

The hybrid system of classical and quantum computing and associated methods will now be discussed generally. The system may include various components and computerized operations, which may be performed on one or more physical devices including a processor and memory. Instructions may be stored in physical memory, which may be performed via the physical processor. The hybrid system of classical and quantum computing for advanced biomedical information may operate one or more of these components interactively with other components for classical and quantum computing operations.

A system and method enabled by this disclosure may advantageously offer a highly scalable, versatile, and robust software-as-a-service (SaaS) platform based on its proprietary technologies. A system and method enabled by this disclosure may democratize informatics and data science by simplifying the analysis of messy big data, fostering fruitful collaboration, breaking inefficient silos, as well as automating complex analytics, machine learning, and artificial intelligence. It may advantageously provide cutting-edge computational workflows and data applications for domains such as biomedical research and clinical developments. Example applications include biomarker discovery for diagnostics and Computer-Aided Drug Design (CADD) for pharmaceuticals.

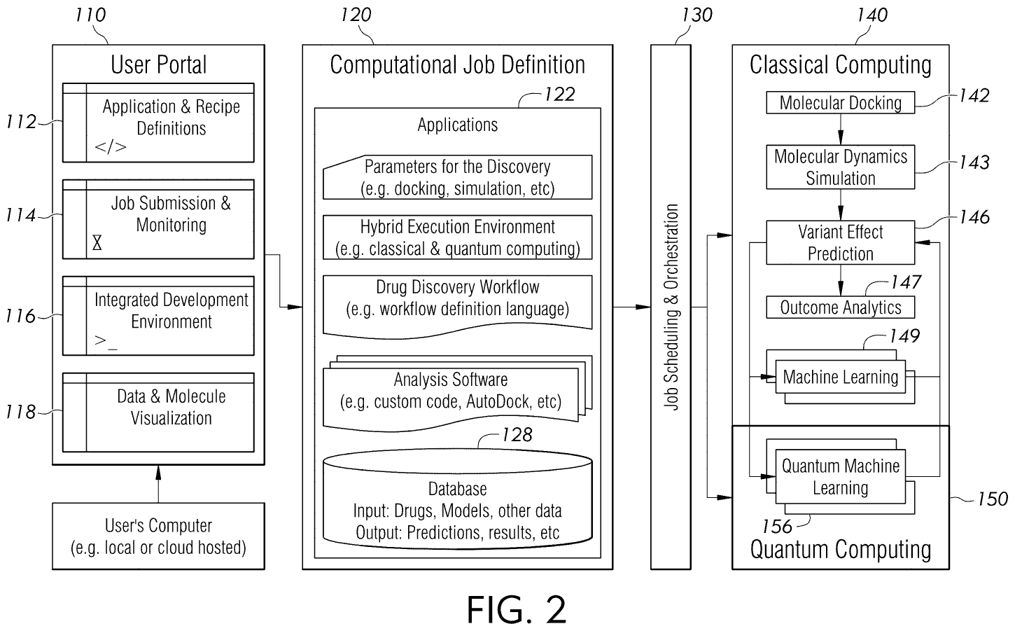

The computing environment will now be discussed in greater detail. FIGS. 1-2 highlight examples of the computing environment, which may also be shown in other figures. A system enabled by this disclosure, may advantageously include a sophisticated end-to-end computational workflow of drug discovery and/or mutation detection comprising multiple interdependent, interconnected, and scatter-and-gather workflows. The workflow may be defined in a platform-agnostic workflow definition language. The workflow execution on a system enabled by this disclosure may leverage multi-core, multi-threads, multi-nodes, and/or other techniques of parallelized processing, advantageously providing highly scalable, computationally efficient, and cost-effective operation, without limitation.

A system enabled by this disclosure may feature one or more computational workflows that include one or multiple computer programs defined by the users and/or the system. The input and output data, the parameters, the workflow definition, the programs used in the workflow, both private and public, and the execution environment may be captured and defined in the system, for example, via an interface 110. This input and data may be analyzed by a job definition aspect 120 (DataLinG), which may be analyzed by software applications 122 (RecApps), which may contribute to versioning, data tracking, and reproducibility as well as for automated reconstruction of the workflows. The software applications 122 may assist with tasks relating to discovery, analysis, data processing, data manipulation, and/or other tasks that would be appreciated by those of skill in the art. The processing jobs for the computing workflows may be scheduled and orchestrated by a system such as enabled by this disclosure and may be performed on classical and quantum computing infrastructures accordingly.

In one embodiment, a system and method enabled by this disclosure may advantageously deploy and run on a cloud computing environment or on premises. It may facilitate execution of arbitrary user-defined computer code, which may be stored in an operatively accessible database 128, ranging from a single program to a distributed computational workflow. A computational workflow can include a number of individual programs and data components that may be virtually linked together and may be performed via computing environment, which may be stored in the database 128. The workflow can be specified by a list of commands or by a workflow definition language specifying the execution sequence and its dependency, including input and output data. Example workflow applications include mutation detection, including for example sequence alignment, alignment recalibration, variant calling, and variant recalibration, as well as RNA expression analysis, including for example sequence mapping, transcription quantification, and differential expression analysis, without limitation.

For both inexperienced computer users and cloud computing experts, a platform enabled by this disclosure may advantageously ease the secured use of public/private credentials for code and data retrieval, computation execution, as well as result upload. In one embodiment, a proprietary module orchestrates and schedules computation from code, data, compute environment and hardware infrastructures, where each can be drawn from different sources via different credentials.

Furthermore, users may benefit from the improvements of a system enabled by this disclosure without being required to develop computational instructions or format data for, and in some cases specifically for, an intended platform, such as may be provided by a system enabled by this disclosure. It can retrieve and upload data from and to different storages of a private infrastructure and/or cloud providers before, during, and/or after computation, as well as make the data shareable among users. It can additionally build a data lineage graph capturing the data origin, the programs used for processing, the parameters and/or environments used by the programs, and the data output.

The execution programs may include code that is stored and retrieved from code repositories. The repositories may include locally stored data and/or data accessible via a network, for example, via a database 128. The execution parameters and the execution environments, such as operating systems and compute environments, may be stored as metadata and linked to a graph or other visual representation, which may be accessible via an interface 110. With the data lineage, a system and method enabled by this disclosure may advantageously be capable of versioning the execution workflow, which may include user-provided code, user-provided imported programs, and data. The versioned workflow may be reconstructed and re-executed for reproducibility.

A system and method enabled by this disclosure may advantageously leverage quantum computers which exploit fundamental quantum physics where classical computing is approaching its operational and practical limits. Quantum computing is increasingly proficient in solving problems that challenge classical computers, for example intrinsically quantum problems such as modeling the quantum states in a molecule with the quantum phenomena of superposition and entanglement. However, classical computers still show high efficiency as a comparatively matured commodity technology stack to solve existing applications such as low-polynomial-time computation and large-throughput data analytics. Therefore, a system and method enabled by this disclosure advantageously employs a hybrid approach that combines the benefits of both classical and quantum computing to achieve what was unachievable in biomedicine under the state of the art prior to development of the novel aspects described throughout this disclosure.

This disclosure is presented in the context of demonstrating and enabling a hybrid computing approach in Computer-Aided Drug Design (CADD), a hyperscale computational pipeline that integrates large-scale genomics and protein structure data into a sophisticated drug discovery workflow. This context is intended to illustrate an application of a system and method enabled by this disclosure and is not intended to limit the scope of the disclosure to only this example. Those of skill in the art will appreciate additional industries and applications that could benefit from a hybrid system and method leveraging classical and quantum computing after having the benefit of this disclosure, which are intended to be included within the scope of this disclosure.

In this illustrative context, a workflow enabled by this disclosure may include highly computationally intensive applications, such as molecular docking 142, molecular dynamics 143, binding affinity prediction 144, variant effect prediction 146, outcome analysis 147, and AI-based lead search and optimization 148, which may be performed as part of a classical machine learning operation 149. Such workloads may, for example, use quantum computing for virtual screening based on pharmacophore modeling, which will potentially unblock the research on a larger range of molecules with larger sizes, structure analysis 152 such as protein folding or structure prediction, molecular comparison and other molecular analyses 154, and quantum machine learning operations 156. Examples of structure analysis include protein folding, without limitation. Examples of molecular analysis include molecular modeling, molecular simulation, molecular comparison, molecular dynamics, and other molecular analytic operations that would be appreciated by a person of skill in the art after having the benefit of this disclosure. The approach also provides a user-friendly interface 110 for users to specify requirements and visualize discovery results. A platform enabled by this disclosure may advantageously leverage both classical and quantum computing, for example, on the cloud so as to make highly advanced computing readily accessible and meaningful to biomedical scientists for solving previously computationally challenging problems.

One illustrative scenario, provided without limitation, facilitates structure-based drug design that may predict potential compounds by applying a quantum virtual screening algorithm for comparing molecules based on different pharmacophore models. Then, in this illustrative scenario, the illustrative system and methods may use classical computing, for example, to build a physical-statistical classifier to predict the effect of variants from hypothetical mutations of the binding site's sequence and perform machine learning and deep learning for lead search and optimization. Generally, the present disclosure enables systems and methods using a hybrid approach technology to not only significantly reduce the time and cost relating to drug discovery to affect a behavior of a biological subject and other computing operations, but also advantageously test prospective efficacies of discovered drugs in the context of mutations of a biological subject and increase the chance for new scientific discoveries such as discovering new drugs or computational breakthroughs.

As will be made apparent throughout this disclosure, a hybrid computational system is provided that may advantageously distribute computational workloads across classical computing 140 and quantum computing 150 environments. The following examples will be discussed in the context of drug discovery to affect a behavior of a biological subject, but those of skill in the art will appreciate additional applications of such systems and method and should not view this disclosure to be limiting to only drug discovery applications.

A system enabled by this disclosure may include a network over which data may be communicated. The network may include wired, wireless, mobile, and/or other data connections that would be appreciated by those of skill in the art. One or more computing environments, which may include one or more classical computing processors and/or quantum computing processors, may be operatively connected via the network. One or more databases may additionally be connected via the network, such as may selectively store and provide access to code, data stores, such as via a database 128, information from various providers, interface elements, user interactions, and/or other data that may assist in the operation of a system and method enabled by this disclosure.

In this illustrative system, a computing environment may be accessible to a user via an interface 110. The computing environment may include components assessable via the network. The computing environment may include and/or provide access to a classical computing processor to perform the classical computing 140 and a quantum computing processor to perform the quantum computing 150. The illustrative system may additionally include memory to store machine-readable instructions, at least part of which may be performed via the computing environment to improve the state of the computer arts, for example, by allowing the system to receive parameters relating to the biological subject.

These instructions may be received via an interface 110, which will be discussed further below. Additional information may be received via the interface 110, for example, credentials, programs, data via I/O operations, definitions, application preferences and recipe definitions 112, job submissions and monitoring criteria 114, custom instructions provided via an integrated development environment 116, inputs for interacting with data and molecule visualization features 118, and/or other information that would be appreciated by skilled artisans. Information may also be accessed, viewed, or otherwise consumed via the interface 110, for example, visualization information, computational results, and/or data output.

The instructions may additionally be processed by job definition aspect 120, where it may be processed by software applications 122. The job definition aspect 120 may, for example, evaluate the parameters to assist in determining a computational application workflow for the discovery operation, for example, docking, simulation, and other operations. The parameters may describe a biological subject in its entirety, a feature of a biological subject such as a binding receptor or other feature, a protein, a molecule, genetic material, and/or other parameters that would be appreciated by those of skill in the art after having the benefit of this disclosure.

The job definition aspect 120 may additionally define a compute workflow to be performed by the computing environment by receiving a screening protocol relating to the biological subject, for example, using a hybrid execution environment for the compute workflow comprising computing tasks. The screening protocol may define aspects of the biological subject to evaluate, goals to accomplish, and other parameters relating to the desired outcome from a system and method enabled by this disclosure.

The job definition aspect 120 may facilitate connecting to a repository, for example via the network, to retrieve data sets relating to the compute workflow. The data sets may include profiles of the biological compound, known drugs and properties relating to the known drugs, protein compositions relating to the biological compound, receptor behavior, known mutations, and other information that could assist with drug discovery.

In some embodiments, a sample of datasets maybe retrieved. This sample may include entries that are randomly selected, curated, identified, suggested from the output of other operations, and/or otherwise designated. Additional samples may be taken later in the computational workflow to further refine the results achieved, supplement the analysis of a prior data set, or otherwise improve the output of a system or method enabled by this disclosure. At least part of the retrieved datasets may be stored in a database 128.

The jobs definition aspect 120 may include an execution environment, for example a hybrid execution environment, to assist with determining whether classical computing 140 or quantum computing 150 may be appropriate for a job or other computational task. For example, the jobs definition aspect 120 may compare the computing tasks and at least part of the data sets for determining whether a likelihood of an advantage, for example a performance advantage and/or cost advantage, is probable for the quantum computing when compared to the classical computing for the computing tasks. The jobs definition aspect 120, and in one example using an execution environment provided by the job definition aspect, the computing tasks may be compared to parts of the retrieved data sets for determining whether a likelihood of an advantage is probable for the quantum computing when compared to the classical computing for the computing tasks.

Compute workflows may be classified into tasks to be accomplished via classical computing or quantum computing with the highest probable efficiency, most economically efficient computation, and/or other factors that will be appreciated by those of skill in the art after having the benefit of this disclosure. For example, the execution environment may assist with identifying a quantum computing task included by the compute workflow to be performed via the quantum computing for which the advantage appears probable and/or favorable and identifying a classical computing task included by the compute workflow to be performed via the classical computing for which the advantage is improbable. Where the advantage appears improbable and/or not favorable, a more traditional classical computing approach may be identified as sufficient, or in some cases more efficient, over the quantum computing model to achieved desired results.

A drug discovery workflow may then be created, which may include determining workflow definition language, as may be specified by the computational job. At least part of the workflow definition language may be provided by a user or other operator, for example, via the interface 110. The job definition aspect 120 may then determine which application, script, tool, or other operation may be best suited to perform the desired computing task and assign the operation to that application.

Once the job definitions aspect 120 has defined the computational workflow to be performed, a job scheduler 130 may distribute the workflow to classical computing 140 and quantum computing 150 aspects of the computing environment. The computing environment may then perform the compute workflow to produce results. Once at least part of the computational workflow is complete, a system and method enabled by this disclosure may organize the results returned by the computing environment to predict the drug discovery. The results may highlight compounds or other drug candidates demonstrating a favorable efficacy to affect the behavior of the biological subject.

In one embodiment, an additional group of software applications 124 may be provided to assist with the operations discussed above. The additional iteration of software applications 124 may include the entire feature set of the first group of software applications 122, may omit one or more features, may include one or more additional features, or may be otherwise configured. Further additional groups may be included, as indicated by blocks labeled RecApps N and RecApps M included by the job definitions aspect 120. By including one or more additional groups of software applications, the operations of the first group may be accelerated via parallel processing, validated via redundant processing, or otherwise assisted.

The classical computing aspect will now be discussed in greater detail. FIGS. 1-2 highlight examples of the classical computing aspect, which may also be shown in other figures. As discussed above and will be further illustrated in the trials and experimentation section below, classical computing tasks may include computations relating to molecular docking, molecular dynamics simulation, binding affinity prediction, variant effect prediction, outcome analysis, and classical machine learning operations, without limitation.

In one example of classical computing tasks, the classical computing may include evaluation of molecular docking properties. A system enabled by this disclosure may select an appropriate tool to evaluate molecular docking properties, for example and without limitation. The molecular docking operation may conduct an initial virtual screening by estimating the noncovalent binding of receptors and ligands. The receptors may be processed using a custom script, for example, removing hetero atom (HETATM) components including ligands, ions, waters, and other components that would be appreciated by skilled artisans. A receptor for docking may then be prepared to add variables to the computational instructions, for example, adding polar hydrogens and Gasteiger charges. Grid boxes for docking may be centered on an active residue and may be set to extend an appropriate number of grid points in each direction. In this example, a lower predicted binding affinity score may predict higher binding affinity between the receptors and ligands.

In one example of classical computing tasks, binding affinity prediction may be calculated, for example, via molecular dynamics simulation. Such simulations may be performed using a selection of various binding affinities, which may be estimated by an application or tool, favored by a machine learning operation, randomly selected, or otherwise identified. Longer molecular dynamics simulations may be performed on selected aspects, for example, ligands.

Topology and coordinate files may be prepared indicating determined trajectories. Selected molecules may be modified with regard to the computing tasks, such as removing water molecules, adding hydrogen bonds, removing non-protein components, and otherwise as would be appreciated by skilled artisans. Processed data may be used as initial protein coordinates. Starting trajectories may be determined for ligands, for example all ligands, for at least one best docked pose. In instances where atom parameters for ligands are not included in a data set, an all-atom force field may be used for selected receptors. Parameters may be generated for ligands. Additionally, for each protein-ligand complex, solvated molecules may be generated using water models. Sodium ions may be added to neutralize the charges of the protein-ligand complexes.

Molecular dynamics simulations may be performed following a standard protocol including minimization, heating, density, equilibration, and production. A Poisson-Boltzmann surface area and the Generalized Born surface area may be calculated. Binding free energy between the ligands and receptors may be estimated. Results may be generated indicating receptors and ligands showing an increased likelihood to bind based on free energy.

In one example of classical computing tasks, variant effect prediction can be calculated. The classical computing tasks may evaluate the effects of a candidate drug on a variation of a biological subject, for example, via interactions between a protein and the candidate drug. For biological subjects based on viral composition, a hybrid physical-statistical classifier may use genomic, structural, and physicochemical features to remove virus incompatible genomic features to human, animal, or other cellular-based subjects, including scale-invariant feature transform (SIFT) score, PPH score, genomic evolutionary rate profiling (GERP) score, germline, somatic, and allele frequency, to assist with building non-species-specific genome compatible classifiers, for example, non-human genome compatible classifiers, as will be understood by those of skill in the art.

In one example of classical computing tasks, lead search may be performed to assist with identifying a lead compound. The lead search may evaluate various drug candidates included in a data set in the context of the biological subject sought to be affected by a drug and/or the behavior of the biological subject to be affected. Lead candidates having a chemical composition indicative of pharmacological or biological activity likely to be therapeutically useful may be selected, highlighted, or otherwise indicated for inclusion in the results or further consideration.

The quantum computing aspect will now be discussed in greater detail. FIGS. 1-2 highlight examples of the quantum computing aspect, which may also be shown in other figures. As discussed above and will be further illustrated in the trials and experimentation section below, quantum computing tasks may include structure analysis, molecular analysis including molecular comparison, and quantum machine learning operations, without limitation.

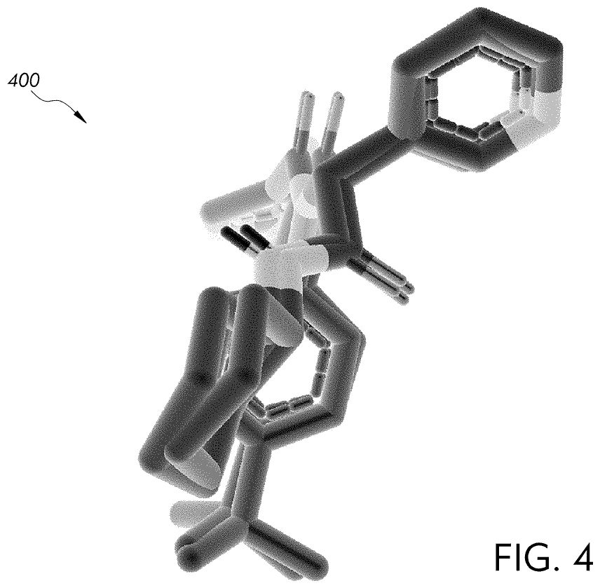

In one example of quantum computing tasks, structure prediction may be performed, for example, to resolve X-ray crystallographic structures of a molecule. Molecules demonstrating positive results may be identified to serve as a benchmark for virtual drug screening of other molecules. Validation may be performed, as would be appreciated by those of skill in the art. A root-mean square deviation (RMSD) may be determined between the experimental structure and best predicted pose of docking, which may be below a defined benchmark to assess the accuracy of docking methodology. The overlay 400 of the crystal structure from trials and experimentation relating to X77 of SARS-CoV-2 and docked pose is shown in FIG. 4. These results may be used to assist other calculations that may be performed via quantum computing and/or classical computing, for example, binding affinity between the receptor and inhibitor.

In one example of quantum computing tasks, molecular analysis, including molecular comparison, molecular simulation, molecular dynamics, and/or other investigations into molecular properties may be performed to gain insight regarding a biological subject. Protocols for a biological subject may be evaluated against a set of ligands, for example, a set of randomly selected ligands and/or previously reported ligands. Results relating to receptors from previous calculations may be compared to show a level at which the ligands' binding affinities to the receptors are correlated and highlight differences that may exist, which might be due to the highly similar but not identical amino acid sequences between variants of a biological subject. Those having skill in the art will appreciate that molecular analysis, including molecular comparison and molecular simulation, may additionally and/or alternatively be performed via classical computing, without limitation.

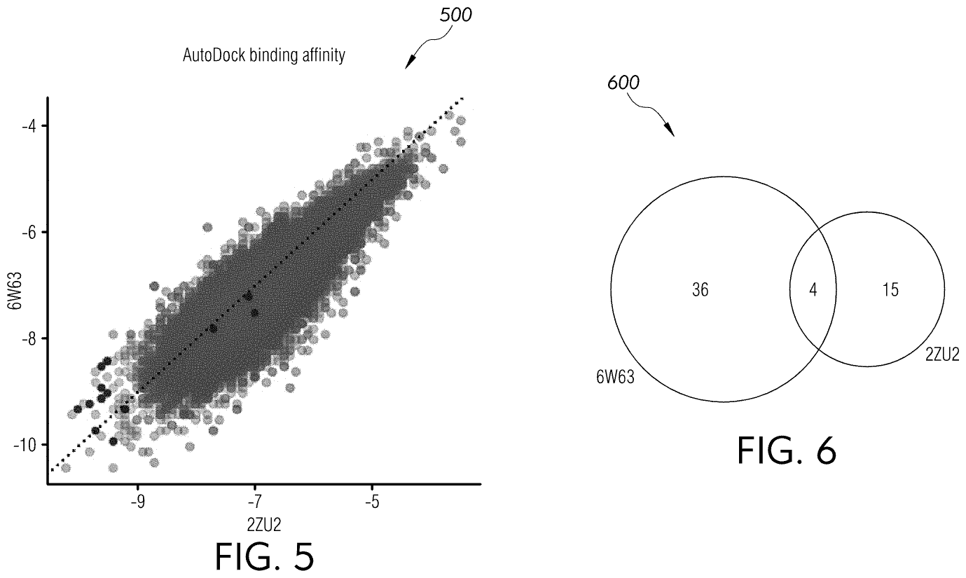

The output may be visualized to a user, for example via an interface, as illustrated by plot 500 of FIG. 5. In this example, 13 ligands are highlighted in plot 500 that shows ligands having a high affinity (<−9.0) to an experimental receptor were not as high to a designated receptor. Additional visualizations of the results may be provided to a user, for example, similar to chart 600 of FIG. 6. Context regarding the actual data presented in FIGS. 4-6 will be discussed in greater detail below along with the trials and experimentation of a system enabled by this disclosure involving drug discovery for the SARS-CoV-2 virus. Additional inhibitors and other features may be evaluated for the impacts from possible variations in the receptor as well.

The machine learning operation will now be discussed in greater detail. FIGS. 1-6 highlight examples of the machine learning operation, which may also be shown in other figures. As discussed throughout this disclosure, the compute workflow may include a machine learning operation to identify a drug having the favorable efficacy. The machine learning operation may advantageously include performance of various machine learning approaches using classical computing and quantum computing capabilities.

In at least one embodiment, the machine learning operation may operate via ensemble machine learning, which may compare and contrast the results of various machine learning operations to increase the collective confidence of the predicted results. In one embodiment, ensemble machine learning may include classical machine learning tasks performed via the classical computing and quantum machine learning tasks performed via the quantum computing. For example, aspects of the job definition aspect may categorize machine learning tasks as having a probability of benefitting from the efficiencies provided via quantum computing. For computing tasks included by the compute workflow indicating a likelihood of an advantage, for example a performance advantage or a cost advantage, is probable and/or favorable from a quantum computing pipeline, quantum machine learning may be applied, as will be understood by those of skill in the art. For computing tasks included by the compute workflow for which a likelihood of the advantage is improbable and/or not favorable from a quantum computing pipeline, classical machine learning may be applied, as will additionally be appreciated by those of skill in the art.

In at least one embodiment, the classical machine learning techniques may include multiple classical machine learning approaches. These approaches may include supervised, unsupervised, reinforcement, meta learning, deep learning, and/or other classical machine learning approaches that would be appreciated by those of skill in the art. For example, classical machine learning approaches may include Random Forest, support vector machine (SVM) using poly kernel (which may feature normalization), SVM using radial basis function (RBF) kernel (which may feature normalization), and other machine learning techniques. The various classical machine learning approaches may be included in the ensemble machine learning approach and may be performed along with the quantum machine learning operations discussed above to increase the quality of results produced. The machine learning operation may additionally assist with identifying a probability of robustness in affecting the behavior of a mutation of the biological subject with approximately the favorable efficacy, which may assist with selecting drug candidates with a high probability of remaining effective when faced with a variant or mutation of a biological subject.

The interface will now be discussed in greater detail. The results of the computational workflow and output of the computing tasks may be provided to a user via an interface 110. The interface 110 may include features to allow a user to interact with a system enabled by this disclosure. In one embodiment, the interface 110 may be provided via a network and may operate as a System-as-a-Service (SaaS). In another embodiment, at least part of the interface 110 may include downloadable and installable aspects.

A user may engage with the interface 110 to provide information and data to a system enabled by this disclosure and retrieve results, data, and visualization from such system. For example, a user may provide parameters relating to a computational workflow to the system. The user may additionally provide credentials, for example, relating to a system enabled by this disclosure, access credentials to a repository storing data sets to be retrieved, licenses for software and tools to be used in the computational workflow, access credentials to remote computing platforms, such as network-based classical and quantum computing platforms, and other credentials as will be appreciated by those of skill in the art. In one embodiment, operations relating to the interface may be performed via classical computing.

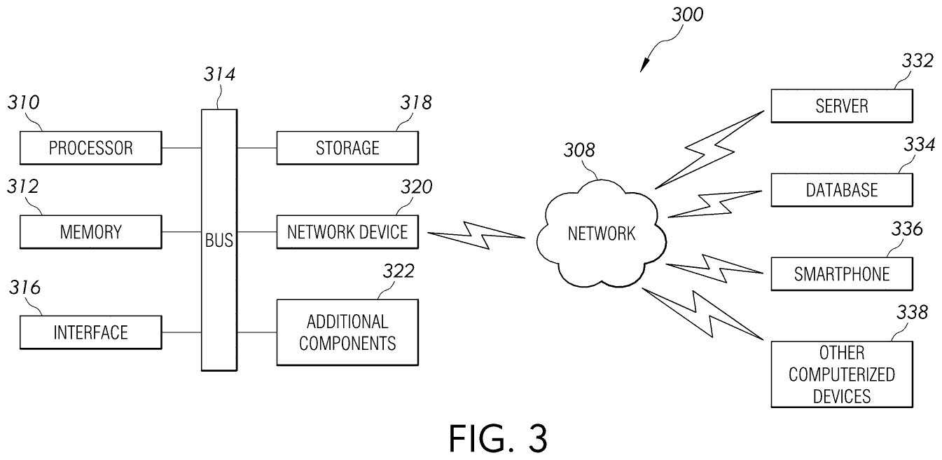

Referring now to FIG. 3, an illustrative computerized device will be discussed, without limitation. Various aspects and functions described in accord with the present disclosure may be implemented as hardware or software on one or more illustrative computerized devices 300 or other computerized devices. There are many examples of illustrative computerized devices 300 currently in use that may be suitable for implementing various classical computing aspects of the present disclosure. Some examples include, among others, network appliances, personal computers, workstations, mainframes, networked clients, servers, media servers, application servers, database servers and web servers. Other examples of illustrative computerized devices 300 may include mobile computing devices, cellular phones, smartphones, tablets, video game devices, personal digital assistants, network equipment, devices involved in commerce such as point of sale equipment and systems, such as handheld scanners, magnetic stripe readers, bar code scanners and their associated illustrative computerized device 300, among others. Additionally, aspects in accord with the present disclosure may be located on a single illustrative computerized device 300 or may be distributed among one or more illustrative computerized devices 300 connected to one or more communication networks.

For example, various aspects and functions may be distributed among one or more illustrative computerized devices 300 configured to provide a service to one or more client computers, or to perform an overall task as part of a distributed system. Additionally, aspects may be performed on a client-server or multi-tier system that includes components distributed among one or more server systems that perform various functions. Thus, the disclosure is not limited to executing on any particular system or group of systems. Further, aspects may be implemented in software, hardware or firmware, or any combination thereof. Thus, aspects in accord with the present disclosure may be implemented within methods, acts, systems, system elements and components using a variety of hardware and software configurations, and the disclosure is not limited to any particular distributed architecture, network, or communication protocol.

FIG. 3 shows a block diagram of an illustrative computerized device 300, in which various aspects and functions in accord with the present disclosure may be practiced. The illustrative computerized device 300 may include one or more illustrative computerized devices 300. The illustrative computerized devices 300 included by the illustrative computerized device may be interconnected by, and may exchange data through, a communication network 308. Data may be communicated via the illustrative computerized device using a wireless and/or wired network connection.

Network 308 may include any communication network through which illustrative computerized devices 300 may exchange data. To exchange data via network 308, systems and/or components of the illustrative computerized device 300 and the network 308 may use various methods, protocols and standards including, among others, Ethernet, Wi-Fi, Bluetooth, TCP/IP, UDP, HTTP, FTP, SNMP, SMS, MMS, SS7, JSON, XML, REST, SOAP, RMI, DCOM, and/or Web Services, without limitation. To ensure data transfer is secure, the systems and/or modules of the illustrative computerized device 300 may transmit data via the network 308 using a variety of security measures including TSL, SSL, or VPN, among other security techniques. The illustrative computerized device 300 may include any number of illustrative computerized devices 300 and/or components, which may be networked using virtually any medium and communication protocol or combination of protocols.

Various aspects and functions in accord with the present disclosure may be implemented as specialized hardware or software executing in one or more illustrative computerized devices 300, including an illustrative computerized device 300 shown in FIG. 3. As depicted, the illustrative computerized device 300 may include a processor 310, memory 312, a bus 314 or other internal communication system, an input/output (I/O) interface 316, a storage system 318, and/or a network communication device 320. Additional devices 322 may be selectively connected to the computerized device via the bus 314. Processor 310, which may include one or more microprocessors or other types of controllers, can perform a series of instructions that result in manipulated data. Processor 310 may be a commercially available processor such as an ARM, x86, Intel Core, Intel Pentium, Motorola PowerPC, SGI MIPS, Sun UltraSPARC, Hewlett-Packard PA-RISC processor, DWAVE, quantum computing processor, or virtually any type of processor or controller as many other processors and controllers are available. As shown, processor 310 may be connected to other system elements, including a memory 312, by bus 314.

The illustrative computerized device 300 may also include a network communication device 320. The network communication device 320 may receive data from other components of the computerized device to be communicated with servers 332, databases 334, smart phones 336, and/or other computerized devices 338 via a network 308. The communication of data may optionally be performed wirelessly. More specifically, without limitation, the network communication device 320 may communicate and relay information from one or more components of the illustrative computerized device 300, or other devices and/or components connected to the computerized device 300, to additional connected devices 332, 334, 336, and/or 338. Connected devices are intended to include, without limitation, data servers, additional computerized devices, mobile computing devices, smart phones, tablet computers, and other electronic devices that may communicate digitally with another device. In one example, the illustrative computerized device 300 may be used as a server to analyze and communicate data between connected devices.

The illustrative computerized device 300 may communicate with one or more connected devices via a communications network 308. The computerized device 300 may communicate over the network 308 by using its network communication device 320. More specifically, the network communication device 320 of the computerized device 300 may communicate with the network communication devices or network controllers of the connected devices. The network 308 may be, for example, the internet. As another example, the network 308 may be a WLAN. However, skilled artisans will appreciate additional networks to be included within the scope of this disclosure, such as intranets, local area networks, wide area networks, peer-to-peer networks, and various other network formats. Additionally, the illustrative computerized device 300 and/or connected devices 332, 334, 336, and/or 338 may communicate over the network 308 via a wired, wireless, or other connection, without limitation.

Memory 312 may be used for storing programs and/or data during operation of the illustrative computerized device 300. Thus, memory 312 may be a relatively high performance, volatile, random access memory such as a dynamic random-access memory (DRAM) or static memory (SRAM). However, memory 312 may include any device for storing data, such as a disk drive or other non-volatile storage device. Various embodiments in accord with the present disclosure can organize memory 312 into particularized and, in some cases, unique structures to perform the aspects and functions of this disclosure.

Components of illustrative computerized device 300 may be coupled by an interconnection element such as bus 314. Bus 314 may include one or more physical busses (for example, busses between components that are integrated within a same machine) but may include any communication coupling between system elements including specialized or standard computing bus technologies such as USB, Thunderbolt, SATA, FireWire, IDE, SCSI, PCI, and InfiniBand. Thus, bus 314 may enable communications (for example, data and instructions) to be exchanged between system components of the illustrative computerized device 300.

The illustrative computerized device 300 also may include one or more interface devices 316 such as input devices, output devices and combination input/output devices. Interface devices 316 may receive input or provide output. More particularly, output devices may render information for external presentation. Input devices may accept information from external sources. Examples of interface devices include, among others, keyboards, bar code scanners, mouse devices, trackballs, magnetic strip readers, microphones, touch screens, printing devices, display screens, speakers, network interface cards, etc. The interface devices 316 allow the illustrative computerized device 300 to exchange information and communicate with external entities, such as users and other systems.

Storage system 318 may include a computer readable and writeable nonvolatile storage medium in which instructions can be stored that define a program to be executed by the processor. Storage system 318 also may include information that is recorded, on or in, the medium, and this information may be processed by the program. More specifically, the information may be stored in one or more data structures specifically configured to conserve storage space or increase data exchange performance. The instructions may be persistently stored as encoded bits or signals, and the instructions may cause a processor to perform any of the functions described by the encoded bits or signals. The medium may, for example, be optical disk, magnetic disk, or flash memory, among others. In operation, processor 310 or some other controller may cause data to be read from the nonvolatile recording medium into another memory, such as the memory 312, that allows for faster access to the information by the processor than does the storage medium included in the storage system 318. The memory may be located in storage system 318 or in memory 312. Processor 310 may manipulate the data within memory 312, and then copy the data to the medium associated with the storage system 318 after processing is completed. A variety of components may manage data movement between the medium and integrated circuit memory element and does not limit the disclosure. Further, the disclosure is not limited to a particular memory system or storage system.

Although the above-described illustrative computerized device is shown by way of example as one type of illustrative computerized device upon which various aspects and functions in accord with the present disclosure may be practiced, aspects of the disclosure are not limited to being implemented on the illustrative computerized device 300 as shown in FIG. 3. Various aspects and functions in accord with the present disclosure may be practiced on one or more computers having components other than that shown in FIG. 3. For instance, the illustrative computerized device 300 may include specially programmed, special-purpose hardware, such as for example, an application-specific integrated circuit (ASIC) tailored to perform a particular operation disclosed in this example. While another embodiment may perform essentially the same function using several general-purpose computing devices running Windows, Linux, Unix, Android, iOS, MAC OS X, or other operating systems on the aforementioned processors and/or specialized computing devices running proprietary hardware and operating systems.

The illustrative computerized device 300 may include an operating system that manages at least a portion of the hardware elements included in illustrative computerized device 300. A processor or controller, such as processor 310, may execute an operating system which may be, among others, an operating system, one of the above-mentioned operating systems, one of many Linux-based operating system distributions, a UNIX operating system, or another operating system that would be apparent to skilled artisans. Many other operating systems may be used, and embodiments are not limited to any particular operating system.

The processor and operating system may work together to define a computing platform for which application programs in high-level programming languages may be written. These component applications may be executable, intermediate (for example, C# or JAVA bytecode) or interpreted code which communicate over a communication network (for example, the Internet) using a communication protocol (for example, TCP/IP). Similarly, aspects in accord with the present disclosure may be implemented using an object-oriented programming language, such as JAVA, C, C++, C#, Python, PHP, Visual Basic.NET, JavaScript, Perl, Ruby, Delphi/Object Pascal, Visual Basic, Objective-C, Swift, MATLAB, PL/SQL, OpenEdge ABL, R, Fortran or other languages that would be apparent to skilled artisans. Other object-oriented programming languages may also be used. Alternatively, assembly, procedural, scripting, or logical programming languages may be used.

Additionally, various aspects and functions in accord with the present disclosure may be implemented in a non-programmed environment (for example, documents created in HTML5, HTML, XML, CSS, JavaScript, or other format that, when viewed in a window of a browser program, render aspects of a graphical-user interface, or perform other functions). Further, various embodiments in accord with the present disclosure may be implemented as programmed or non-programmed elements, or any combination thereof. For example, a web page may be implemented using HTML while a data object called from within the web page may be written in C++. Thus, the disclosure is not limited to a specific programming language and any suitable programming language could also be used.

An illustrative computerized device included within an embodiment may perform functions outside the scope of the disclosure. For instance, aspects of the system may be implemented using an existing commercial product, such as, for example, Database Management Systems such as a SQL Server available from Microsoft of Redmond, Wash., Oracle Database or MySQL from Oracle of Austin, Tex., or integration software such as WebSphere middleware from IBM of Armonk, N.Y.

In operation, a method may be provided for discovering drugs showing efficacy in affecting the behavior of a biological subject. Those of skill in the art will appreciate that the following methods are provided to illustrate an embodiment of the disclosure and should not be viewed as limiting the disclosure to only those methods or aspects. Skilled artisans will appreciate additional methods within the scope and spirit of the disclosure for performing the operations provided by the examples below after having the benefit of this disclosure. Such additional methods are intended to be included by this disclosure.

In one illustrative method, steps may be performed to encourage drug discovery that may affect a behavior of a biological subject. The method may be performed using a computing environment that includes classical computing and quantum computing capabilities. In this illustrative method, parameters may be received relating to the biological subject. The parameters may describe a biological subject in its entirety, a feature of a biological subject such as a binding receptor or other feature, a protein, a molecule, genetic material, and/or other parameters that would be appreciated by those of skill in the art after having the benefit of this disclosure. In another step, the method may define a compute workflow to be performed by the computing environment by receiving a screening protocol relating to the biological subject, the compute workflow comprising computing tasks.

The method may include retrieving data sets from a repository relating to the compute workflow. At least part of the computing tasks may then be compared to at least part of the retrieved data sets for determining a likelihood that an advantage could exist for the quantum computing compared to classical computing for the computing tasks. For example, the comparison may determine that an advantage exists for the quantum computing based on a significant increase in performance. In another example, the comparison may detect a cost advantage for using classical computing in light of a marginal performance advantage being provided by the quantum computing, without limitation. The method may then continue with classifying compute workflows into tasks to be accomplished via classical computing or quantum computing.

The method may include distributing a quantum computing task to be performed via the quantum computing if included by or recommended based on the likelihood of the advantage by the compute workflow. For example, this step may include identifying a quantum computing task included by the compute workflow to be performed via the quantum computing for which the likelihood of an advantage appears probable and/or favorable. The method may also include distributing a classical computing task to be performed via the classical computing if included by or recommended based on the likelihood of the advantage by the compute workflow. For example, this step may include identifying a classical computing task included by the compute workflow to be performed via the classical computing for which the likelihood of an advantage for using quantum computing appears improbable and/or is not favorable, signaling that the more traditional classical computing approach will be sufficient, or in some cases more efficient, having a favorable advantage to achieve desired results.

The method may then perform the compute workflow via the computing environment, including classical computing and quantum computing, to produce results. As discussed below, the results may be indicative of drug candidates showing a significant probability of affecting the behavior of a biological subject. This step may further include identifying a drug having the favorable efficacy using a machine learning operation, such as discussed above. Additionally, a probability of robustness in affecting the behavior of a mutation of the biological subject with approximately the favorable efficacy may be identified, advantageously enhancing the value of drug candidates returned during the execution of the drug discovery compute workflow. In this step, machine learning operations may be performed via ensemble machine learning, wherein classical machine learning tasks are performed via the classical computing, and quantum machine learning tasks are performed via the quantum machine learning.

The results from the compute workflow may then be organized and returned by the computing environment to predict the drug discovery demonstrating a favorable efficacy to affect the behavior of the biological subject. The results may be presented to a user via the interface. Additionally, data may be retrievable by the user via the interface or otherwise, for example, via direct download or programmatic retrieval.

Trials and Experimentation

This disclosure is further described by the following example trial and experimentation data, provided without limitation. To validate the system and method enabled by this disclosure, a trial was conducted to demonstrate a hybrid computing approach enabled by a system and method discussed throughout this disclosure, namely in the context a HypaCADD implantation for Computer-Aided Drug Design.

The HypaCADD experimental system is provided as a hyperscale computational pipeline that integrates large-scale genomics and protein structure data into a sophisticated drug discovery workflow of highly computationally intensive applications, such as molecular docking, binding affinity prediction, molecular dynamics, and AI-based lead search and optimization.

In this study, a hybrid system of classical and quantum computing (see FIG. 1) and improved method and process of drug discovery (see FIG. 2) was used. A sequential virtual screening protocol was applied including molecular docking and molecular dynamics simulations, to about 30,000 ligands and identified 2 promising candidates for the new coronavirus, SARS-CoV-2, of COVID-19. A hybrid physical-statistical classifier was built capable of predicting the effect of variants from hypothetical and real mutations in receptor sequences that can guide the lead search and optimization. The novel approach of this experimentation and disclosure is believed to not only significantly reduce the time and cost for drug development, but also largely increase the chance and robustness in discovering new drugs.

Datasets

A drug dataset (features: 3D, Ref/Mid pHs, Drug-like, In-stock) from ZINC in PDBQT format was downloaded from https://zinc.docking.org/, comprising a total of more than 10 million compounds. The X-ray crystallized 3D structure of 3CL^{pro} of HCoV-229E (PDB code: 2ZU2)2 and 3CL^{pro} of SARS-CoV-2 of COVID-19 (PDB code 6W63) were downloaded from https://www.rcsb.org/. 2ZU2 was used as the receptor to validate the screening methodology of the experimentation in SARS-CoV and 6W63 was used as the receptor to identify lead compounds for SARS-CoV-2.

Both receptor files were downloaded in PDB format, without limitation. Real mutations in SARS-COV-2 on nsp5 3CL^{pro} were collected from https://covidcg.org/.

Molecular Docking

AutoDock Vina was chosen as the docking tool to conduct the initial virtual screening by estimating the noncovalent binding of receptors and ligands. The receptors were processed using a custom script, removing “HETATM” components including ligands, ions, and waters and protonating His41 to the neutral state at the epsilon nitrogen (Nε2). Then the preparation of a receptor for docking was finished by a script in AutoDock Tools, where the polar hydrogens and Gasteiger charges were added and the PDB file were converted to PDBQT format. The grid boxes for docking were centered on the active His41 residue and were set to extend 26 grid points in each direction. Lower predicted binding affinity score correlated with the higher binding affinity between the receptors and ligands.

Molecular Dynamics Simulation

Molecular dynamics simulations (0.4 ns) were performed with various binding affinities estimated by AutoDock. Longer molecular dynamics simulations (10 ns) were performed on selected ligands, including the ligand named X77 that was co-crystallized with SARS-CoV-2 3CL^{pro}.

All topology and coordinate files in this experimentation were prepared using Ambertools 2.0. For all trajectories in this experimentation, the protein PDB files were processed using the “pdb4amber” function in Ambertools to remove water molecules, add hydrogen bonds, and remove non-protein components. Then the processed PDB files were used as initial protein coordinates. The starting trajectories for all ligands were the best docked poses generated by AutoDock Vina. Since ﬀ14SB does not include atom parameters for ligands, the all-atom force field AMBER (ﬀ14SB) was only used for receptors and ANTECHAMBER was applied to generate parameters for all ligands in this experimentation, according to General Amber Force Field 2 (GAFF2). For each protein-ligand complex in this experimentation, the dry and solvated molecules were generated using PBradii mbondi3 and TIP3P water models. At the end, sodium ions were added to neutralize the charges of the protein-ligand complexes.

Molecular dynamics simulations were performed using sander.MPI of the AMBER packages, following a standard protocol including minimization, heating, density, equilibration, and production. Detailed input parameters were provided for molecular dynamics simulation. The Poisson-Boltzmann surface area (MM-PBSA) and the Generalized Born surface area (MM-GBSA) were calculated using a script (MMPBSA.py) in AMBER. Both MM-PBSA and MM-GBSA were used for the estimation of the binding free energy between the ligands and receptors. The lower MM-GBSA/MM-PBSA score is, the easier for receptors and ligands to bind based on free energy.

A hybrid physical-statistical classifier for classical computers was used to predict the impacts of SNVs on protein-drug interactions. The classifier selected uses genomic, structural, and physicochemical features. Since the classifier focused on human proteins, some genomic features were not suitable for non-human subjects such as viruses. In this study, those virus incompatible genomic features, including SIFT score, PPH score, GERP score, germline, somatic, and allele frequency, were removed in an attempt to build non-human genome compatible classifiers.

In the interest of further improving model prediction accuracy, the experimentation utilized an ensemble machine learning method by building multiple machine learning models, including Random Forest, SVM using ploy kernel, SVM using RBF kernel, and quantum machine learning (QML). Quantum machine learning was based on binary classifiers with a margin of loss to classify data. Each classifier is implemented on variational circuits. The QML model was implemented using Pennylane and ran on an AMS Bracket service, which is a cloud-hosted service for quantum computing.

Results and Method Validation

From here, the experimentation followed an established approach to identify lead compounds, which have shown promises as inhibitors of 3CL^{pro} in SARS-CoV. To obtain confidence in the screening method of the experimentation, performance was validated with 13 out of their 19 reported ligands which had identical ZINC IDs in the version of the drug dataset used. Beside the 13 ligands, 29,995 randomly selected ligands from the drug dataset were also used for method validation.

The same molecular docking method, AutoDock Vina, was applied to the 13 ligands from the established approach and 29,995 randomly selected ligands, docking against the 2ZU2 receptor, a 3D structure of 3CL^{pro} in SARS-CoV. All the 13 reported ligands had binding affinities less than −7 kcal/mol and 8 of them had binding affinities less than −9.5 kcal/mol (Table 1), which was the screening criteria used in the established approach.

TABLE 1
Binding affinities from molecular
docking using the 2ZU2 receptor.

| Ligands | Affinity |
|---|---|
| ZINC000002426719 | −7 |
| ZINC000009104621 | −10 |
| ZINC000009346433 | −7.1 |
| ZINC000009411012 | −7.7 |
| ZINC000009477134 | −9.6 |
| ZINC000012550995 | −9.6 |
| ZINC000012597223 | −9.6 |
| ZINC000012697660 | −9.5 |
| ZINC000012798320 | −9.8 |
| ZINC000015999133 | −9.4 |
| ZINC000020130947 | −9.5 |
| ZINC000032983195 | −9.2 |
| ZINC000035829976 | −9.7 |

Compared to the 29,995 random ligands, the reported 13 ligands were highly enriched with ligands that can bind to the 2ZU2 receptor with high affinity (Fisher's Exact test p-value <2.2e-16) (Table 2), indicating we were able to identify high-quality candidates in our initial screening for potential lead compounds.

TABLE 2
The number of ligands satisfying binding affinity
cutoffs among 13 ligands from known techniques
and 29,984 randomly selected ligands.

| Affinity criteria | paper | random |
|---|---|---|
| >−9.5 | 5 | 29984 |
| =<−9.5 | 8 | 11 |

Fisher's Exact test: p-value < 2.2e−16

Calculating MM-GBSA score is a more sophisticated, but computationally intensive method to assess the free energy of binding between receptors and ligands. The experimentation used an MM-GBSA calculation implemented by the AMBER software. The AMBER MM-GBSA calculations were applied to a manageable subset of the aforementioned ligands with varying binding affinities predicted by AutoDock Vina.

Specifically, based on Table 2, 19 ligands with binding affinities <=−9.5 kcal/mol and 13 ligands (5 from the established approach and 8 randomly selected) with binding affinities >9.5 were processed with MM-GBSA. MM-GBSA successfully processed 18 of the 32 ligands, as shown in Table 3.

TABLE 3
Free energy of binding predicted by MM-GBSA for a collection
of 18 ligands in gas phase (MM-GBSA Gas), solvent phase
(MM-GBSA solv) and combined (MM-GBSA total). Each method
evaluated configurations to report lower scores for
higher binding tendency. Ligands with affinity <−9.0
kcal/mol were labeled in light gray.

| | | MM- | MM- | MM- |
|---|---|---|---|---|
| | Autodock | GBSA | GBSA | GBSA |
| Ligands | affinity | Total | Gas | solv |
| ZINC000002484659 | −4.7 | −12.24 | −43.00 | 30.76 |
| ZINC000005121078 | −5.4 | −18.88 | −32.31 | −18.88 |
| ZINC000002473650 | −5.6 | −21.08 | −34.67 | 13.58 |
| ZINC000008386858 | −5.7 | −17.59 | −31.83 | 14.24 |
| ZINC000009411012 | −7.7 | 568.05 | 534.47 | 33.58 |
| ZINC000032983195 | −9.2 | −46.71 | −66.99 | 20.28 |
| ZINC000101766867 | −9.5 | −49.45 | −66.89 | 17.44 |
| ZINC000247300899 | −9.5 | −22.63 | −37.32 | 14.69 |
| ZINC000020130947 | −9.5 | −34.29 | −44.80 | 10.51 |
| ZINC000012697660 | −9.5 | −37.81 | −43.60 | 5.79 |
| ZINC000057129043 | −9.6 | −32.58 | −49.14 | 16.56 |
| ZINC000015953686 | −9.6 | −33.16 | −47.73 | 14.57 |
| ZINC000012550995 | −9.6 | −35.25 | −45.59 | 10.34 |
| ZINC000035829976 | −9.7 | −40.30 | −49.47 | 9.17 |
| ZINC000015959761 | −9.7 | −35.49 | −49.26 | 13.77 |
| ZINC000016001299 | −9.8 | −44.46 | −71.90 | 27.44 |
| ZINC000012798320 | −9.8 | −33.51 | −58.47 | 24.96 |
| ZINC000016020583 | −10.2 | −42.10 | −58.29 | 16.19 |

The comparison of AutoDock affinity scores and MM-GBSA scores (Table 3) showed that ligands with lower AutoDock affinity score (higher binding affinity) had lower MM-GBSA score (easier to bind based on free energy). The results demonstrated that MM-GBSA's predicted results were in general consistent with the AutoDock's, indicating that the more sophisticated MM-GBSA can potentially help filter candidates for high quality.

Overall, results from the experimentation have demonstrated the virtual screening and simulation combined can reliably serve as the foundation for predicting lead compounds for further optimization and selection.

Defining benchmarks and accuracy of virtual screening for SARS-CoV-2 3CL^{pro}

Recently, there have been several resolved X-ray crystallographic structures of SARS-CoV-2 3CL^{pro}; however, most of those structures are complex with an irreversible substrate-like inhibitor. At the time of this study, only one structure (PDB code: 6W63) represented SARS-CoV-2 3CL^{pro} with a reversible dipeptide inhibitor (X77). This structure and inhibitor could serve as a benchmark for virtual drug screening for SARS-CoV-2 3CL^{pro}. To validate the docking method for SARS-CoV-2 3CL^{pro}, AutoDock Vina and MM-GBSA were applied to the receptor and inhibitor X77 in the 6W63 structure. The root-mean square deviation (RMSD) between the experimental structure of X77 and best predicted pose of docking by AutoDock Vina was 0.814 Å, which was below the well-defined 2 Å benchmark to assess the accuracy of docking methodology. The overlay of the crystal structure of X77 and docked pose is shown in FIG. 4.

The binding affinity and MM-GBSA between the receptor and inhibitor of 6W63 were −8.3 kcal/mol and −43.20 kcal/mol, respectively.

Molecular Docking and Molecular Dynamics for SARS-CoV-2 3CL^{pro}

Confirming with established approach and benchmarking on the 6W63 structure provided confidence to use AutoDock Vina and MM-GBSA as high throughput tools for virtual screening.

The experimentation further applied the protocol to SARS-CoV-2 3CL^{pro} against the 29,995 randomly selected ligands and 13 previously reported ligands, as aforementioned. Comparison of AutoDock results of the two receptors showed the ligands' binding affinities to 2ZU2 and 6W63 were highly correlated but some differences existed, as seen in FIGS. 5-6, which might be due to the highly similar but not identical amino acid sequences between SARS-Cov 3CL^{pro} and SARS-Cov-2 3CL^{pro}. For example, the 13 ligands highlighted on FIG. 5 shows that ligands which were having a high affinity (<−9.0) to 2ZU2 were not as high to 6W63. There were 40 ligands (Table 4) with binding affinity <=−9.5 kcal/mol for SARS-Cov-2 3CL^{pro}, of which only 4 overlapped with those for SARS-Cov shown in FIG. 6.

TABLE 4
a) Binding affinities (<=−9.5 kcal/mol) from molecular docking
using the 6W63 receptor and free energy of binding predicted by MM-GBSA
for a subset of ligands in gas phase (Gas) and combining gas and solvent (total).

| | Auto | Auto | | | | | |
|---|---|---|---|---|---|---|---|
| | Dock | Dock | | | | | |
| | affinity | affinity | | mmGBSA_6W63 | | mmPBSA_6W63 | |
| | with | with | | (0.4 ns) | | (0.4 ns) | |
| ligand | 2ZU2 | 6W63 | category | GAS | Total | GAS | Total |
| X77 | | −8.3 | control | −51.73 | −43.20 | −51.73 | −6.03 |
| ZINC000001869737 | −7.6 | −9.5 | random | | | | |
| ZINC000001877004 | −9.2 | −9.5 | random | | | | |
| ZINC000002704038 | −9.1 | −9.8 | random | −47.63 | −33.61 | −47.63 | −2.39 |
| ZINC000009332191 | −9.1 | −10 | random | | | | |
| ZINC000009498932 | −9.2 | −9.9 | random | −42.05 | −24.79 | −42.05 | 6.66 |
| ZINC000009644901 | −8 | −9.5 | random | −61.89 | −44.32 | −61.89 | −8.55 |
| ZINC000010054369 | −8.2 | −9.7 | random | −51.24 | −34.19 | −51.24 | −1.78 |
| ZINC000011695427 | −8.5 | −9.5 | random | | | | |
| ZINC000012376777 | −7.8 | −9.5 | random | | | | |
| ZINC000012444302 | −8.1 | −10 | random | | | | |
| ZINC000015953686 | −9.6 | −9.8 | random | 664.25 | 681.83 | 664.25 | 694.15 |
| ZINC000015999133 | −9.4 | −9.9 | paper | | | | |
| ZINC000016001299 | −9.8 | −10.1 | random | | | | |
| ZINC000016020583 | −10.2 | −10.4 | random | −71.81 | −57.87 | −71.81 | −22.27 |
| ZINC000018180793 | −8.6 | −9.5 | random | | | | |
| ZINC000018235600 | −8.4 | −9.6 | random | | | | |
| ZINC000018266226 | −9.4 | −10.4 | random | −49.86 | −37.78 | −49.86 | −4.59 |
| ZINC000019238929 | −8.7 | −9.8 | random | | | | |
| ZINC000032205245 | −9.1 | −9.7 | random | −38.68 | −31.61 | −38.68 | −2.08 |
| ZINC000033308623 | −8.5 | −9.7 | random | | | | |
| ZINC000033435744 | −9 | −9.5 | random | | | | |
| ZINC000034758692 | −9.3 | −9.6 | random | 799.57 | 827.51 | 799.57 | 850.65 |
| ZINC000034812138 | −9.2 | −9.7 | random | | | | |
| ZINC000035424775 | −9.1 | −9.6 | random | −46.05 | −32.35 | −46.05 | −2.48 |
| ZINC000035829976 | −9.7 | −9.7 | paper | −65.03 | −52.87 | −65.03 | −10.18 |
| ZINC000035851646 | −8.8 | −9.5 | random | | | | |
| ZINC000036707984 | −7.7 | −9.7 | random | | | | |
| ZINC000082049652 | −9 | −9.7 | random | −66.77 | −33.40 | −66.77 | −7.05 |
| ZINC000085875174 | −8.4 | −9.6 | random | | | | |
| ZINC000096114211 | −9 | −9.5 | random | −42.83 | −27.57 | −42.83 | −0.82 |
| ZINC000096114284 | −8.8 | −10.1 | random | | | | |
| ZINC000096114628 | −8.3 | −9.7 | random | 493.06 | 519.45 | 493.06 | 546.18 |
| ZINC000096115318 | −8.6 | −9.7 | random | | | | |
| ZINC000096331475 | −9.3 | −9.8 | random | 506.49 | 506.49 | 506.49 | 542.14 |
| ZINC000096444131 | −8.9 | −10.1 | random | | | | |
| ZINC000096833037 | −9 | −9.8 | random | −68.65 | −47.99 | −68.65 | −10.94 |
| ZINC000097480050 | −7.7 | −9.7 | random | −77.40 | −52.35 | −77.40 | −19.85 |
| ZINC000105321111 | −9.2 | −9.6 | random | | | | |
| ZINC000230129735 | −8.7 | −10.3 | random | | | | |
| ZINC000245240291 | −8.8 | −9.5 | random | | | | |

TABLE 4
b) the selected 10 ligands for 10 ns simulation. Category
indicates the selection source of the ligand.

| | Auto | Auto | | | | | |
|---|---|---|---|---|---|---|---|
| | Dock | Dock | | | | | |
| | affinity | affinity | | mmGBSA_6W63 | | mmPBSA_6W63 | |
| | with | with | | (10 ns) | | (10 ns) | |
| ligand | 2ZU2 | 6W63 | category | GAS | Total | GAS | Total |
| X77 | | −8.3 | control | −71.30 | −52.54 | −71.30 | −14.29 |
| ZINC000005486523 | −5 | −5.7 | random | −34.38 | −29.14 | −34.38 | −9.23 |
| ZINC000072117658 | −6.6 | −6.6 | random | −62.22 | −9.41 | −62.22 | −1.77 |
| ZINC000009411012 | −7.7 | −7.8 | paper | | | | |
| ZINC000020130947 | −9.5 | −8.4 | paper | −50.89 | −33.16 | −50.89 | 1.54 |
| ZINC000002467880 | −9.7 | −8.6 | random | −69.12 | −52.41 | −69.12 | −16.24 |
| ZINC000009644901 | −8 | −9.5 | random | −59.44 | −44.19 | −59.44 | −7.98 |
| ZINC000034758692 | −9.3 | −9.6 | random | 828.91 | 845.89 | 828.91 | 845.89 |
| ZINC000009498932 | −9.2 | −9.9 | random | −61.66 | −44.52 | −61.66 | −4.44 |
| ZINC000016020583 | −10.2 | −10.4 | random | −74.42 | −64.73 | −74.42 | −28.21 |
| | Auto | Auto | | | | | |
| | Dock | Dock | | | | | |
| | affinity | affinity | | mmGBSA_6W63 | | mmPBSA_6W63 | |
| | with | with | | (0.4 ns) | | (0.4 ns) | |
| ligand | 2ZU2 | 6W63 | category | GAS | Total | GAS | Total |
| X77 | | −8.3 | control | −51.73 | −43.20 | −51.73 | −6.03 |
| ZINC000005486523 | −5 | −5.7 | random | −34.36 | −29.90 | −34.36 | −8.72 |
| ZINC000072117658 | −6.6 | −6.6 | random | −72.83 | 4.05 | −72.83 | 6.95 |
| ZINC000009411012 | −7.7 | −7.8 | paper | 844.19 | 862.79 | 844.19 | 886.08 |
| ZINC000020130947 | −9.5 | −8.4 | paper | −66.38 | −40.41 | −66.38 | −5.06 |
| ZINC000002467880 | −9.7 | −8.6 | random | −29.76 | −20.39 | −29.76 | −0.68 |
| ZINC000009644901 | −8 | −9.5 | random | −61.89 | −44.32 | −61.89 | −8.55 |
| ZINC000034758692 | −9.3 | −9.6 | random | 799.57 | 827.51 | 799.57 | 850.65 |
| ZINC000009498932 | −9.2 | −9.9 | random | −42.05 | −24.79 | −42.05 | 6.66 |
| ZINC000016020583 | −10.2 | −10.4 | random | −71.81 | −57.87 | −71.81 | −22.27 |

Furthermore, MM-GBSA ran successfully on 17 of the 40 ligands (binding affinity <=−9.5 kcal/mol) plus X77 with a 0.4 ns simulation (Table 4a). Ideally, a longer simulation such as 10 ns would have more stable and accurate results, though it would take much longer runtime. For the proof of concept of this study, the experimentation ran a 10 ns simulation with the 6W63 receptor on a selected set of 10 ligands, i.e.: 1) X77, which served as a control; 2) the ligand, i.e. ZINC000016020583, in the aforementioned 17 ligands which had both the free energies in “gas” and in “total” less than the results of X77 from the 10 ns simulation; 3) a random set of 8 ligands of any binding affinity from docking. Results in Table 4b shows that ZINC000016020583 still had lower MM-GBSA scores than X77 and another ligand, ZINC000002467880, also had a comparable MM-GBSA score with X77, indicating these two ligands could have higher or comparable binding affinity to the 6W63 receptor. Therefore, they were selected as the potential lead compounds in this study. Along with the reference inhibitor X77, they were evaluated for the impacts from possible variations in the receptor as well.

See FIG. 4 regarding the overlap of the crystal structure of X77 and docked pose. See also FIGS. 5-6 regarding comparison of AutoDock binding affinity. In FIG. 5, a scatter plot of AutoDock is presented illustrating a binding affinity between the 2ZU2 and 6W63 receptors. The highlighted dots represent the 13 reported ligands. In FIG. 6, a Venn diagram is presented illustrating the number of ligands with AutoDock affinity <=−9.5 kcal/mol for the 6W63 and 2ZU2 receptors.

Evaluation of the Impacts of Virus Variations on the Interactions of the Lead Compounds with SARS-CoV-2 3CL^{pro}

GenoDock is a hybrid Physical-Statistical classifier to predict the impacts of variants on protein-drug interactions using genomic, structural, and chemical features. However, due to the focus on human proteins, some of the features used in the published GenoDock classifier were human-specific and not applicable to viral genomes. In order to make the GenoDock framework applicable to non-human genomes such as viruses and to further improve the predictive performance, the experimentation made several modifications and improvements including removing human-specific features, feature normalization, and making use of an ensemble method. Table 5 demonstrated that the new virus-genome compatible classifier with Random Forest (RF) implementation had comparable performance compared to the original GenoDock classifier with Random Forest implementation based on a 10-fold cross-validation.

TABLE 5
Comparison of the original GenoDock classifier and new classifier,
which is virus-genome compatible after removing human-specific
features. Both classifiers were implemented using the Random
Forest machine learning model. The numbers in parenthesis are
standard deviation of the performance scores.

| | | remove human- |
|---|---|---|
| | original | specific features |
| Accuracy | 0.965 (0.005) | 0.964 (0.006) |
| F1 | 0.701 (0.040) | 0.705 (0.047) |
| recall | 0.640 (0.050) | 0.663 (0.061) |
| precision | 0.779 (0.054) | 0.756 (0.056) |

A commonly used technique in machine learning is “ensemble method”, which utilizes multiple machine learning models to obtain a better predictive performance than an individual model. Therefore, the experimentation further implemented diverse virus-genome compatible machine learning models to predict variants' impact on protein-drug interactions, including SVM based on poly kernel, SVM based on RBF kernel, and quantum machine learning (QML) model. 3-fold cross-validation results (Table 6) indicated feature normalization was necessary for SVM models, where the models with feature normalization had better predictive performance than the ones without feature normalization. The QML (described above) also showed high concordance with classical machine learning models (QML vs RF: 95.92%; QML vs SVM poly: 95.92%; QML vs SVM RBF: 96.60%), indicating its reliability.

TABLE 6
Comparison of SVM based classifiers. The classifier without normalization failed
to detect true positives, therefore the F1, recall, and precision were all 0's.
The numbers in parenthesis are standard deviations of the performance scores.

| | SVM poly kernel | | SVM rbf kernel | |
|---|---|---|---|---|
| | w/o | with | w/o | with |
| | normalization | normalization | normalization | normalization |
| Accuracy | 0.935 (0.000) | 0.950 (0.002) | 0.935 (0.000) | 0.948 (0.003) |
| F1 | 0 (0) | 0.539 (0.022) | 0 (0) | 0.513 (0.016) |
| recall | 0 (0) | 0.450 (0.021) | 0 (0) | 0.423 (0.009) |
| precision | 0 (0) | 0.674 (0.030) | 0 (0) | 0.654 (0.041) |

As of January 2021, 99 amino acid variations had been detected on SARS-CoV-2 3CL^{pro}. To evaluate the impact of variations on the interaction of SARS-CoV-2 3CL^{pro} and potential drugs, multiple machine learning models, i.e. Random Forest, SVM using poly kernel (with feature normalization), SVM using RBF kernel (with feature normalization), and QML, were applied to the three aforementioned ligands, namely ZINC000016020583, ZINC000002467880, and X77. Ensemble predictions from the four classifiers of the experimentation were based on a majority rule (Table 7a). It predicted in total 9 mutations, 3 per ligand, that would have an impact on the 3 input ligands. Specifically, X77 was predicted to be impacted by variations at position 43 (ILE:VAL) and 188 (ARG:LYS & ARG:SER) on its interaction with SARS-CoV-2 3CL^{pro}; ZINC000002467880 was by variations at positions 168 (PRO:SER) and 188 (ARG:LYS & ARG:SER); where as ZINC000016020583 was by variations 168 (PRO:SER), 184 (PRO:SER) and 188 (ARG:SER). Variation at position 43 was not predicted to have an impact on both ZINC000016020583 and ZINC000002467880 by any model, as opposed to X77.

TABLE 7a
Protein-drug interactions interruption prediction for variations on SARS-CoV-2 3CL^{pro} (variations
that were predicted as positive by majority rule, i.e. in at least 3 models, were shown).

| | | | | | within | | SVM poly |
|---|---|---|---|---|---|---|---|
| | | | | | binding | Random | kernel with |
| receptor | position | ref | alt | ligand | site | Forest | normalization |
| 6W63 | 43 | ILE | VAL | X77 | 1 | 1 | 0 |
| 6W63 | 188 | ARG | LYS | X77 | 1 | 1 | 1 |
| 6W63 | 188 | ARG | SER | X77 | 1 | 1 | 1 |
| 6W63 | 168 | PRO | SER | ZINC000002467880 | 1 | 1 | 1 |
| 6W63 | 188 | ARG | LYS | ZINC000002467880 | 1 | 1 | 1 |
| 6W63 | 188 | ARG | SER | ZINC000002467880 | 1 | 1 | 1 |
| 6W63 | 168 | PRO | SER | ZINC000016020583 | 1 | 1 | 1 |
| 6W63 | 184 | PRO | SER | ZINC000016020583 | 1 | 1 | 1 |
| 6W63 | 188 | ARG | SER | ZINC000016020583 | 1 | 0 | 1 |

| | | | | | SVM rbf | SVM-inspired | |
|---|---|---|---|---|---|---|---|
| | | | | | kernel with | quantum machine | |
| receptor | position | ref | alt | ligand | normalization | learning | Ensemble |
| 6W63 | 43 | ILE | VAL | X77 | 1 | 1 | 3 |
| 6W63 | 188 | ARG | LYS | X77 | 1 | 0 | 3 |
| 6W63 | 188 | ARG | SER | X77 | 1 | 1 | 4 |
| 6W63 | 168 | PRO | SER | ZINC000002467880 | 1 | 1 | 4 |
| 6W63 | 188 | ARG | LYS | ZINC000002467880 | 1 | 1 | 4 |
| 6W63 | 188 | ARG | SER | ZINC000002467880 | 1 | 1 | 4 |
| 6W63 | 168 | PRO | SER | ZINC000016020583 | 1 | 0 | 3 |
| 6W63 | 184 | PRO | SER | ZINC000016020583 | 1 | 0 | 3 |
| 6W63 | 188 | ARG | SER | ZINC000016020583 | 1 | 1 | 3 |

TABLE 7b
Variation at position 43 was not predicted as interrupted
by any model for both ZINC000016020583 and ZINC000002467880

| | | | | | within | | SVM poly |
|---|---|---|---|---|---|---|---|
| | | | | | binding | Random | kernel with |
| receptor | position | ref | alt | ligand | site | Forest | normalization |
| 6W63 | 43 | ILE | VAL | X77 | 1 | 1 | 0 |
| 6W63 | 43 | ILE | VAL | ZINC000002467880 | 0 | 0 | 0 |
| 6W63 | 43 | ILE | VAL | ZINC000016020583 | 0 | 0 | 0 |

| | | | | | SVM rbf | SVM-inspired | |
|---|---|---|---|---|---|---|---|
| | | | | | kernel with | quantum machine | |
| receptor | position | ref | alt | ligand | normalization | learning | Ensemble |
| 6W63 | 43 | ILE | VAL | X77 | 1 | 1 | 3 |
| 6W63 | 43 | ILE | VAL | ZINC000002467880 | 0 | 0 | 0 |
| 6W63 | 43 | ILE | VAL | ZINC000016020583 | 0 | 0 | 0 |

The results indicate that even though three known mutations at amino acid positions 43 (ILE:VAL) and 188 (ARG:LYS and ARG:SER) could have an impact on X77, they would have a different effect on ZINC000016020583 and ZINC000002467880. For example, both 43 (ILE:VAL) and 188 (ARG:LYS) may not impact ZINC000016020583 as much.

Discussion Regarding the Experimentation

The experimentation established a hybrid system of classical and quantum computing to advance Computer-Aided Drug Design. It utilizes a number of well-established virtual screening methods, such as molecular docking and molecular dynamics simulations, to identify lead compounds using a novel implementation and workflow. It leverages machine learning to build an improved physical-statistical classifier capable of predicting the effect of variants from hypothetical and real mutations in receptor sequences of any genome so as to provide insights into drug efficacy. From screening to lead identification and to variant effect prediction, an approach demonstrated by the experimentation can work not only in classical computing, but also in conjunction with quantum computing to go beyond the current limits of classical computers. The experimentation demonstrated its performance and capability by applying the system to viral genomes and with quantum machine learning (QML).

Antiviral drugs targeting SARS-CoV-2 3CL^{pro} could help to fight against the COVID-19 pandemic. As a proof of concept, the experimentation validated screening methods with the SARS-CoV 3CL^{pro} protein. The experimentation then applied a system and method enabled by this disclosure to the SARS-CoV-2 3CL^{pro} protein in COVID-19, where two lead compounds, ZINC000016020583 and ZINC000002467880, were identified as potential inhibitors from ˜30,000 compounds subsampled from a drug dataset of over 11 million compounds.

As viruses mutate frequently, so is the new coronavirus. It is essential to predict the effect of the mutants efficiently and reliably on any target drugs in the design process and to know the efficacy and alternatives of a candidate drug. The experimentation therefore further applied an improved method of variant effect prediction to the identified compounds by using machine learning and both classical and quantum computing techniques. The experimentation results showed indicative and insightful results based on ˜100 known mutations thus far, proving the utility of an approach enabled by this disclosure for an advanced, promising, and robust drug design.

While various aspects have been described in the above disclosure, the description of this disclosure is intended to illustrate and not limit the scope of the invention. The invention is defined by the scope of the appended claims and not the illustrations and examples provided in the above disclosure. Skilled artisans will appreciate additional aspects of the invention, which may be realized in alternative embodiments, after having the benefit of the above disclosure. Other aspects, advantages, embodiments, and modifications are within the scope of the following claims.

What is claimed is:

1. A hybrid computational system using classical computing and quantum computing for drug discovery to affect a behavior of a biological subject comprising:
a network over which data is communicated;
a computing environment accessible via the network comprising:
a classical computing processor to perform the classical computing, and
a quantum computing processor to perform the quantum computing;
a memory on which is stored machine-readable instructions to:
(a) receive parameters relating to the biological subject;
(b) define a compute workflow to be performed by the computing environment by receiving a screening protocol relating to the biological subject, the compute workflow comprising computing tasks;
(c) connect to a repository via the network to retrieve data sets relating to the compute workflow;
(d) selectively compare at least part of the computing tasks and at least part of the data sets for determining a likelihood of an advantage for the quantum computing compared to the classical computing for the computing tasks;
(e) distribute a quantum computing task to be performed via the quantum computing if included by or recommended based on the likelihood of the advantage by the compute workflow;
(f) distribute a classical computing task to be performed via the classical computing if included by or recommended based on the likelihood of the advantage by the compute workflow;
(g) perform the compute workflow via the computing environment to produce results;
(h) organize the results returned by the computing environment to predict the drug discovery demonstrating a favorable efficacy to affect the behavior of the biological subject.

2. The system of claim 1, wherein the compute workflow comprises a machine learning operation to identify a drug having the favorable efficacy.

3. The system of claim 2, wherein the machine learning operation further identifies a probability of robustness in affecting the behavior of a mutation of the biological subject with approximately the favorable efficacy.

4. The system of claim 2, wherein the machine learning operation operates via ensemble machine learning comprising:
classical machine learning tasks performed via the classical computing; and/or
quantum machine learning tasks performed via the quantum machine learning.

5. The system of claim 1, wherein the classical computing tasks comprise molecular docking.

6. The system of claim 1, wherein the classical computing tasks comprise binding affinity prediction.

7. The system of claim 1, wherein the classical computing tasks comprise variant effect prediction.

8. The system of claim 1, wherein the classical computing tasks comprise lead search.

9. The system of claim 1, wherein the quantum computing tasks comprise structure analysis.

10. The system of claim 1, wherein the quantum computing tasks comprise molecular analysis.

11. The system of claim 1, further comprising an interface;
wherein the parameters are received via the interface; and
wherein at least some of the results are presented via the interface.

12. A method for drug discovery to affect a behavior of a biological subject performed via a hybrid computational system using a computer environment to perform classical computing and quantum computing, the method comprising:
(a) receiving parameters relating to the biological subject;
(b) defining a compute workflow to be performed by the computing environment by receiving a screening protocol relating to the biological subject, the compute workflow comprising computing tasks;
(c) retrieving data sets from a repository relating to the compute workflow;
(d) selectively comparing at least part of the computing tasks and at least part of the data sets for determining a likelihood of an advantage for the quantum computing compared to the classical computing for the computing tasks;
(e) distributing a quantum computing task to be performed via the quantum computing if included by or recommended based on the likelihood of the advantage by the compute workflow;
(f) distributing a classical computing task to be performed via the classical computing if included by or recommended based on the likelihood of the advantage by the compute workflow;
(g) performing the compute workflow via the computing environment to produce results;
(h) organizing the results returned by the computing environment to predict the drug discovery demonstrating a favorable efficacy to affect the behavior of the biological subject;
wherein the parameters are received via an interface; and
wherein at least some of the results are presented via the interface.

13. The method of claim 12, wherein the computing environment is operable over a network; and
wherein data is communicated via the network.

14. The method of claim 1, wherein the compute workflow comprises a machine learning operation; and
wherein step (g) further comprises:
(1) identifying a drug having the favorable efficacy via the machine learning operation.

15. The method of claim 14, wherein step (g) further comprises:
(2) identifying a probability of robustness in affecting the behavior of a mutation of the biological subject with approximately the favorable efficacy.

16. The method of claim 14, wherein step (g) further comprises:
(3) performing the machine learning operation via ensemble machine learning, wherein classical machine learning tasks are performed via the classical computing, and/or quantum machine learning tasks are performed via the quantum machine learning.

17. The method of claim 12, wherein the classical computing tasks comprise:
molecular docking;
binding affinity prediction;
variant effect prediction; and/or
comprise lead search.

18. The method of claim 12, wherein the quantum computing tasks comprise:
structure analysis; and/or
molecular analysis.

19. A method for drug discovery to affect a behavior of a biological subject performed via a hybrid computational system using a computer environment to perform classical computing and quantum computing, the method comprising:
(a) receiving parameters relating to the biological subject;
(b) defining a compute workflow to be performed by the computing environment by receiving a screening protocol relating to the biological subject, the compute workflow comprising computing tasks;
(c) retrieving data sets from a repository relating to the compute workflow;
(d) selectively comparing at least part of the computing tasks and at least part of the data sets for determining a likelihood of an advantage for the quantum computing compared to the classical computing for the computing tasks;
(e) distributing a quantum computing task to be performed via the quantum computing if included by or recommended based on the likelihood of the advantage by the compute workflow, the quantum computing tasks comprising:
structure analysis, and/or
molecular analysis;
(f) distributing a classical computing task to be performed via the classical computing if included by or recommended based on the likelihood of the advantage by the compute workflow, the classical computing tasks comprising:
molecular docking,
binding affinity prediction,
variant effect prediction, and/or
comprise lead search;
(g) performing the compute workflow via the computing environment to produce results, further comprising:
(1) identifying a drug having a favorable efficacy in affecting the behavior of the biological subject via the machine learning operation, and
(2) identifying a probability of robustness in affecting the behavior of a mutation of the biological subject with the favorable efficacy;
(h) organizing the results returned by the computing environment to predict the drug discovery demonstrating the favorable efficacy.

20. The method of claim 19, wherein the parameters are received via an interface, and
wherein at least some of the results are presented via the interface.
